(19) United States
(12) Patent Application Publication
(10) Pub. No.: US 2026/0316949 A1
(43) Pub. Date: Oct. 15, 2026

(54) PASSIVATION SYSTEM

(71) Applicant: AerSale, Inc., Doral, FL (US)

(72) Inventors: Iso Nezaj, Weston, FL (US); Gayan Henadirage, Naperville, IL (US); Nathan Regan, Placentia, CA (US); Matthew Zimmerman, Franksville, WI (US); Matthew Wilfong, Algonquin, IL (US)

(73) Assignee: AerSale, Inc., Doral, FL (US)

(21) Appl. No.: 19/642,567

(22) Filed: Apr. 8, 2026

Related U.S. Application Data
(60) Provisional application No. 63/786,171, filed on Apr. 9, 2025.

(51) Int. Cl. B64D 37/32; B64D 37/00; B64D 37/02; B64F 5/10; G01N 33/28; H01B 13/00

(52) U.S. Cl. CPC B64D 37/32; B64D 37/005; B64D 37/02; B64F 5/10; G01N 33/28; H01B 13/0013

(57) ABSTRACT

A wiring passivation system is provided for passivating an electrical wire harness. The system may have a support member installable inside a controlled environment of a vehicle structure and attachable to the vehicle structure. The support member may support a passivation tray that holds a foam block in physical contact with an electrical wire of the electrical wire harness. The foam block is compressed by the passivation tray against a vehicle structure so that the electrical wire is isolated from fluids inside the controlled environment.

### CROSS-REFERENCE TO RELATED APPLICATIONS

This application is based upon and claims the benefit of and priority to U.S. Provisional Patent Application No. 63/786,171 entitled “TANK PASSIVATION SYSTEM,” filed on Apr. 9, 2025, the entire content of which is incorporated by reference herein.

### FIELD

The present disclosure generally relates to a system, device, or apparatus for protecting wiring, particularly sensor wiring in a fuel tank.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may be inventions.

Frequently wiring is routed through environments where an electrical fault in the wiring could become hazardous. For instance, many aircraft are equipped with a fuel quantity indicating system that uses a sensor with electrical wiring to sense an amount of fuel in an aircraft fuel tank. Because fuel is combustible, there is a need for a system to ameliorate combustion risk in an event of a fault in the electrical wiring. However, various prior solutions present non-ideal tradeoffs such as unacceptably diminishing a total usable fuel volume associated with the fuel tank or presenting maintenance or retrofit difficulties. Further, evolving regulatory standards present additional requirements that exceed those of originally manufactured equipment, necessitating systems, devices, and apparatuses suitable for retrofit installations.

### SUMMARY

A wiring passivation system is provided. The system may be for passivating an electrical wire harness. The system may include a support member, a passivation tray, and a foam block. The support member may be installable inside a controlled environment of a vehicle structure. The support member may be attachable to the vehicle structure with at least one attachment member. The passivation tray may include at least one sheet metal flange attachable to the support member. The foam block may be retained in physical contact with the electrical wire harness by the passivation tray. The foam block may be compressed by the passivation tray in compressive loading between the passivation tray and the vehicle structure.

In various instances, one or more further features are provided. In various instances, the passivation tray is attached to the support member by a further at least one attachment member. In various embodiments, the further at least one attachment member is an electrically non-conductive mechanical connector. In various embodiments, the at least one attachment member is an electrically non-conductive mechanical connector. In various embodiments, the foam block includes a plurality of foam pieces having interlocking shapes. In various embodiments, the passivation tray is a single piece of sheet metal having a plurality of bends. In various embodiments, the vehicle structure is an airplane. In various embodiments, the controlled environment is an airplane fuel tank.

A method is provided. The method may be a method of passivating an electrical wire harness. The method may include installing a support member inside a controlled environment of a vehicle structure by attaching the support member to the vehicle structure with at least one attachment member. The method may include attaching a passivation tray to the support member. The passivation tray may include at least one sheet metal flange. The method may include inserting a foam block into the passivation tray. The method may include retaining the foam block in physical contact with the electrical wire harness by the passivation tray, wherein the foam block is compressed by the passivation tray in compressive loading between the passivation tray and the vehicle structure.

In various instances, one or more further features are provided. In various embodiments, the inserting the foam block into the passivation tray is performed before the attaching the passivation tray to the support member. In various embodiments, the inserting the foam block into the passivation tray is performed after the attaching the passivation tray to the support member. In various embodiments, the method further includes interlocking a plurality of foam blocks having interlocking shapes to form the foam block, wherein the foam block comprises the plurality of foam blocks having interlocking shapes. In various embodiments, the passivation tray is a single piece of sheet metal having a plurality of bends. In various embodiments, the vehicle structure is an airplane. In various embodiments, the controlled environment is an airplane fuel tank.

A fuel quantity indicating probe system is provided for a vehicle fuel tank having a vehicle structure. The system may include a support member attached by at least one attachment member to the vehicle structure and extending through a controlled environment. The controlled environment may have an interior space bounded by the vehicle structure, the interior space configured to retain a flammable fluid. The system may include a passivation tray including at least one sheet metal flange sheet attached to the support member and providing a channel to receive a foam block. The system may include a sensor to detect an amount of the flammable fluid in the interior space. The system may include an electrical wire harness connected to the sensor to provide data or signals corresponding to the amount from the sensor to an indicator disposed outside of the controlled environment. The system may include the foam block, retained by the passivation tray in physical contact against the electrical wire harness, wherein the passivation tray presses the foam block in compressive loading between the passivation tray and the vehicle structure.

In various instances, one or more further features are provided. In various embodiments, the passivation tray is attached to the support member by a further at least one attachment member. In various embodiments, the further at least one attachment member is an electrically non-conductive mechanical connector. In various embodiments, the at least one attachment member is an electrically non-conductive mechanical connector. In various embodiments, the foam block has a plurality of foam pieces having interlocking shapes.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the following detailed description and claims in connection with the following drawings. While the drawings illustrate various embodiments employing the principles described herein, the drawings do not limit the scope of the claims.

FIG. 1 illustrates an example wiring passivation system, in accordance with various embodiments;

FIG. 2 illustrates a method of passivating an electrical wire harness, in accordance with various embodiments;

FIG. 3 illustrates an aircraft having a vehicle structure that is a center fuel tank, in accordance with various embodiments;

FIG. 4 shows a detailed view of aspects of the wiring passivation system installed in the vehicle structure, in accordance with various embodiments;

FIG. 5 shows a subset of features illustrated in FIG. 4 for additional clarity, in accordance with various embodiments;

FIG. 6 shows an end view of the support member of FIG. 5, in accordance with various embodiments;

FIGS. 7A and 7B show detailed views of aspects of the support member illustrated in FIG. 5, in accordance with various embodiments;

FIG. 8 shows an opposite end view of the support member of FIG. 5, in accordance with various embodiments;

FIG. 9 shows a passivation tray attached to the support member by attachment members, as well as the support member attached to the vehicle structure by attachment members, in accordance with various embodiments;

FIGS. 10A-B illustrate multiple passivation trays attached together and supported by a same support member, in accordance with various embodiments;

FIG. 11A illustrates a first subassembly of passivation trays, in accordance with various embodiments;

FIG. 11B shows the first subassembly, with foam blocks illustrated as transparent rectangles, in accordance with various embodiments;

FIG. 12 illustrates an example foam block defining a channel to receive wiring, in accordance with various embodiments;

FIG. 13 illustrates myriad arbitrary shapes available for passivation trays, in accordance with various embodiments;

FIG. 14 shows an illustration of passivation trays from FIG. 13 in a looking-up orientation, in accordance with various embodiments;

FIG. 15 shows an illustration of passivation trays from FIG. 13 in a looking-forward orientation, in accordance with various embodiments;

FIG. 16 shows an illustration of passivation trays from FIG. 13 detailing a tray-to-tray joint, in accordance with various embodiments;

FIG. 17 shows an illustration having details of a portion of FIG. 11B, and in particular, installation of foam blocks, in accordance with various embodiments;

FIG. 18 shows a portion of the wiring passivation system having a crawlspace through a bulkhead, in accordance with various embodiments;

FIG. 19 depicts an illustration of similar features as FIG. 18 but from a looking-forward direction and showing the crawlspace opening, in accordance with various embodiments;

FIG. 20 shows an illustration of an assembly depicted in FIG. 17, in accordance with various embodiments;

FIG. 21 shows an illustration of the crawlspace through the bulkhead, in accordance with various embodiments;

FIG. 22 shows an illustration of aspects of the crawlspace but with the foam blocks omitted, in accordance with various embodiments;

FIGS. 23 and 24 show detail views of various passivation trays having a right-angle bent flange to accommodate passage of a wiring in a right-angle corner of a controlled environment of a vehicle structure, such as to accommodate placement of a fuel level sensing probe, in accordance with various embodiments;

FIG. 25 shows an aircraft having a vehicle structure that is a wing fuel tank, in accordance with various embodiments;

FIG. 26 shows a detailed view of aspects of the wiring passivation system installed in the vehicle structure, in accordance with various embodiments;

FIG. 27 shows an electrical wire harness with a probe extending through a vehicle structure, in accordance with various embodiments;

FIG. 28 provides a view of additional aspects of an electrical wire harness extending through foam block supported by a passivation tray in a wing fuel tank, in accordance with various embodiments;

FIG. 29 illustrates a detail view of a forward/aft substructure, in accordance with various embodiments;

FIG. 30 shows a detail view of various other features including flanges and structures to attach passivation trays in a desired location, in accordance with various embodiments;

FIG. 31 shows a detail view of an inboard/outboard substructure, in accordance with various embodiments;

FIG. 32 depicts a view of further features including flanges and structures to attach passivation trays in desired locations shown from a looking-aft direction, in accordance with various embodiments;

FIG. 33 depicts a view of further features including flanges and structures to attach passivation trays in desired locations shown from a looking-forward direction, in accordance with various embodiments;

FIG. 34 depicts a view of further features including flanges and structures to attach passivation trays in connection with a float valve assembly for sensing a fuel level in a fuel tank, in accordance with various embodiments;

FIG. 35 depicts a view showing yet further details of foam blocks and passivation trays, in accordance with various embodiments; and

FIG. 36 depicts a view showing gusset installations in connection with mounting passivation trays, in accordance with various embodiments.

### DETAILED DESCRIPTION

The following detailed description of various embodiments herein refers to the accompanying drawings, which show various embodiments by way of illustration. While these various embodiments are described in sufficient detail to enable those skilled in the art to practice the disclosure, it should be understood that other embodiments may be realized and that changes may be made without departing from the scope of the disclosure. Thus, the detailed description herein is presented for purposes of illustration only and not of limitation. Furthermore, any reference to singular includes plural embodiments, and any reference to more than one component or step may include a singular embodiment or step. Also, any reference to attached, fixed, connected, or the like may include permanent, removable, temporary, partial, full or any other possible attachment option. Additionally, any reference to without contact (or similar phrases) may also include reduced contact or minimal contact. It should also be understood that unless specifically stated otherwise, references to “a,” “an” or “the” may include one or more than one and that reference to an item in the singular may also include the item in the plural. Further, all ranges may include upper and lower values and all ranges and ratio limits disclosed herein may be combined.

Disclosed herein is a fuel tank flammability reduction system that facilitates compliance with the Fuel Quantity Indication System (FQIS) Airworthiness Directive (AD). The FQIS AD requires amelioration of potential ignition sources inside a fuel tank FQIS system in various fuel tanks. By implementing systems and methods herein, the FQIS AD is complied with. A system provided herein ameliorates risk and limits the available oxygen that can ignite fuel vapors and prevents sparks.

Advantageously, this system and method have nothing to turn on, no required pilot monitoring, no moving parts, no pressure lines, no relief valves, no detonation sensing devices, no functional components to fail, maintain, monitor, or need other periodic inspections, no onboard high pressure storage vessels to protect. Moreover, the system and method minimally reduces fuel capacity, does not add significant weight to an airframe, and has a lengthy installation life limit.

In various embodiments, wiring and other structures in an aircraft fuel tank are passivated by encapsulation in a foam body. Various foam bodies (e.g., blocks) may be pre-formed and may be installed throughout various locations in an aircraft fuel tank to encapsulate and/or at least partially fluidically isolate the wiring from liquid and/or vapor fuel in the fuel tank. For instance, in various embodiments, foam is provided around FQIS wiring, preventing spark ignition, and yet refraining from filling an entire tank with reticulated polyurethan foam and positioned in a non-contacting relation with the wiring, segregating the wiring. Advantageously, the system herein may be operable without additionally introducing any wiring, probes or other related equipment into an aircraft fuel tank.

Various straps, baskets with intercostals, flanges, hangers, glue, clicklocks, and other mechanisms are implemented to secure and support the system. Non-limiting embodiments are illustrated herein.

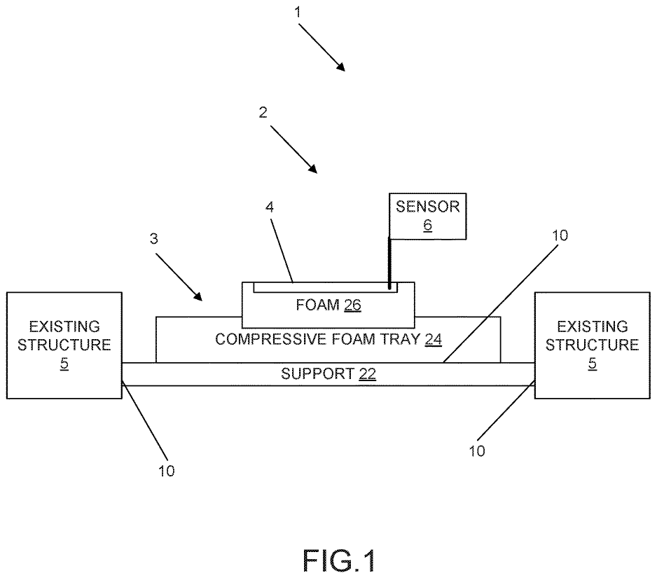

With reference to FIG. 1, a wiring passivation system 2 is provided. The wiring passivation system 2 may be provided inside a vehicle structure 5. In various embodiments the vehicle structure 5 is a fuel tank. For instance, the vehicle structure 5 may be a fuel tank of an aircraft. The vehicle structure 5 may be a center fuel tank of an aircraft. The vehicle structure 5 may be a fuselage fuel tank of an aircraft. The vehicle structure 5 may be a wing fuel tank of an aircraft. In various embodiments, the tank is a fluid container that is installed into an aircraft structure. In various embodiments, the tank is integrally formed in an aircraft structure, such as being defined by various stringers, ribs, bulkheads, and other structure members of an aircraft.

The vehicle structure 5 may include a controlled environment 3. The controlled environment 3 may be a volume of space defined and bounded by the vehicle structure 5. The controlled environment 3 may be a space having fluids such as liquids or gasses. In various instances, the fluids are flammable. In various instances, the controlled environment 3 may be a volume of space configured to hold aviation fuel for use by an aircraft.

The vehicle structure 5 may include wiring 4. For instance, wiring 4 may connect to one or more sensor that monitors a characteristic of the controlled environment 3, such as to measure a quantity of fuel therein. The wiring 4 may connect to other aircraft systems, such as an aircraft data bus, or to an indicating system configured to indicate human or machine-readable data corresponding to the characteristic that the one or more sensor monitors. The wiring 4 may be referred to as electrical wiring, an electrical wire harness, or a harness assembly elsewhere herein.

The wiring 4 may be subject to an airworthiness directive requiring additional passivation or protection. For instance, electrical wiring in an aircraft fuel tank may present various risks in connection with various fault conditions. For instance, electrical wiring may become hot or damaged, insulation may melt or become damaged, faults may provide a fuel ignition source, or other harmful outcomes may occur.

Consequently, a wiring passivation system 2 may be provided. The wiring passivation system may at least partially encapsulate the electrical wiring so that electrical faults are isolated from fluid in the controlled environment 3. The wiring passivation system 2 may include foam blocks 26. The foam blocks 26 may be positioned in physical contact with the wiring 4. For instance, the foam block 26 may provide a channel through which the wiring passes. The foam blocks 26 may press against the wiring, constraining the wiring between a vehicle structure 5, such as a bulkhead, stringer, rib, or other airframe component, so that a combination of the foam blocks 26 and the vehicle structure 5 isolates the wiring from exposure to the fluid in the controlled environment 3. In various instances, the foam blocks 26 are cut into shapes that interlock with adjacent foam blocks 26. Moreover, the foam blocks may be cut so that the interlocking joints between adjacent foam blocks 26 have discontinuities, irregular shapes, or otherwise lie in more than one plane. In this manner, inadvertent exposure of wiring 4 to the fluid in the controlled environment 3 may be mitigated. For instance, in a fault condition of the electrical wiring 4, local increased gas pressure such as that associated with an arc or heating, may cause the foam blocks to tend to separate. By providing interlocking joints between adjacent foam blocks 26 that lie in more than one plane, have irregular shapes, or have discontinuities, this pressure change may be dissipated without providing a fluid ingress path from the controlled environment 3 to the wiring 4.

The wiring passivation system 2 may include a passivation tray 24. The passivation tray 24 is a structure that supports the foam blocks 26 in a desired orientation. The passivation tray 24 may provide a channel that holds the foam blocks on one or more side, while positioning an aspect of the foam blocks in a desired contacting relation or a desired proximate but non-contacting relation, with the wiring 4. In various embodiments, the foam blocks are in a desired non-contacting relation proximate to the wiring. The foam blocks may create an area around the wire segregated from the rest of a fuel tank so that a spark, combustion, or other fault form the wire is suppressed/mitigated from igniting other content of the fuel tank. For instance, the passivation tray 24 may have a generally U-shaped profile or incomplete trapezoid profile to hold foam blocks between aspects of the passivation tray 24 in a pressure or frictional relation, and to also exert force conducted by the blocks and reacted by a vehicle structure 5 so that the blocks are pressed against the existing structure with the wiring 4 between the foam blocks 26 and the vehicle structure 5. In various embodiments, the passivation tray 24 is a bent sheet metal flange. In various embodiments, the foam blocks are slidably inserted into the passivation tray 24 and the passivation tray is sized to compress the foam blocks to retain the foam blocks in the passivation tray 24.

The wiring passivation system 2 may include a support member 22. The support member 22 may be a rod, flange, extrusion, or any other member that joins the passivation tray 24 to the vehicle structure 5, sot that the passivation tray 24 is oriented in proper relation to the vehicle structure 5 to provide desired protection to the wiring. The support member 22 may comprise a member extending between two distinct aspects of the vehicle structure 5 and connected at multiple locations. The support member 22 may comprise a member cantilevered from an aspect of the vehicle structure 5. The support member 22 may be connected to the vehicle structure 5 by a mechanical fastener. The mechanical fastener may have a dielectric component so that the mechanical fastener does not provide electrical continuity between the vehicle structure 5 and the support member 22. The mechanical fastener may be made of a non-conductive material so that the mechanical fastener does not provide electrical continuity between the vehicle structure 5 and the support member 22. In various embodiments, the mechanical fastener is a Click-Loc® fastener available from Moeller Aerospace (Moeller, Mfg. Co., Inc. of Wixom, Michigan). In further embodiments, the mechanical fastener may be a glue, a weld, an interlocking fitment, or any mechanism of joining in a fixed physical relation.

The support member 22 may be connected to the passivation tray 24 by a similar fastener. The support member 22 may be connected to the passivation tray 24 by a mechanical fastener. The mechanical fastener may have a dielectric component so that the mechanical fastener does not provide electrical continuity between the passivation tray 24 and the support member 22. The mechanical fastener may be made of a non-conductive material so that the mechanical fastener does not provide electrical continuity between the passivation tray 24 and the support member 22. In various embodiments, the mechanical fastener is a Click-Loc® fastener available from Moeller Aerospace (Moeller, Mfg. Co., Inc. of Wixom, Michigan). In further embodiments, the mechanical fastener may be a glue, a weld, an interlocking fitment, or any mechanism of joining in a fixed physical relation. Moreover, while reference is made to fasteners, such as Click-Loc® fasteners in several instances, one may appreciate that the fasteners may be used in various same or different instances in different embodiments. Such fasteners may be used to affix various stanchions and metallic frame elements for the support members and passivation trays to the physical fuel tank structure. Consequently, these fasteners may provide a mechanism to affix and hod foam to needed surfaces without creating a need to electrically ground the structures. These fasteners may provide support for pressure baffling in an event of a fault, ignition or the like within an interior chamber proximate to a wiring that is created by foam blocks all emplaced together.

Thus, a wiring passivation system 2 for passivating an electrical wire harness 4 may have a support member 22, a passivation tray 24, and a foam block 26. The support member 22 is installable inside a controlled environment 3 of a vehicle structure 5. The support member 22 is attachable to the vehicle structure 5 with at least one attachment member 10. The passivation tray 24 may include at least one sheet metal flange attachable to the support member 22. The wiring passivation system 2 may have a foam block 26 retained in physical contact with the electrical wire harness 4 by the passivation tray 24, wherein the foam block 26 is compressed by the passivation tray 24 in compressive loading between the passivation tray 24 and the vehicle structure 5.

The wiring passivation system 2 may have various aspects or features. For instance, the passivation tray 24 may be attached to the support member 22 by a further at least one attachment member 10. The further at least one attachment member 10 may be an electrically non-conductive mechanical connector. Similarly, the at least one attachment member 10 may be an electrically non-conductive mechanical connector.

The foam block 26 may have a plurality of foam pieces having interlocking shapes. The passivation tray 24 may be a single piece of sheet metal having a plurality of bends. The vehicle structure 5 may be an airplane. The controlled environment 3 may be an airplane fuel tank.

Stated another way, a fuel quantity indicating probe system 1 may be provided for a vehicle fuel tank (vehicle structure 5) having a vehicle structure 5. The system may include a support member 22 attached by at least one attachment member 10 to the vehicle structure 5 and extending through a controlled environment 3 comprising an interior space bounded by the vehicle structure 5, the interior space configured to retain a flammable fluid. The system may include a passivation tray 24 comprising at least one sheet metal flange sheet attached to the support member 22 and providing a channel to receive a foam block 26. The system may include a sensor 6. The sensor 6 may be to detect an amount of the flammable fluid in the interior space of the controlled environment 3. An electrical wire harness (wiring 4) may be connected to the sensor 6 to provide data or signals corresponding to the amount from the sensor 6 to an indicator disposed outside of the controlled environment. The system may include the foam block 26, retained by the passivation tray 24 in physical contact against the electrical wire harness (wiring 4), wherein the passivation tray presses the foam block in compressive loading between the passivation tray 24 and the vehicle structure 5.

The passivation tray 24 may be attached to the support member 22 by a further at least one attachment member 10. The further at least one attachment member may be an electrically non-conductive mechanical connector. The at least one attachment member 10 may be an electrically non-conductive mechanical connector. The foam block 26 comprises a plurality of foam pieces having interlocking shapes.

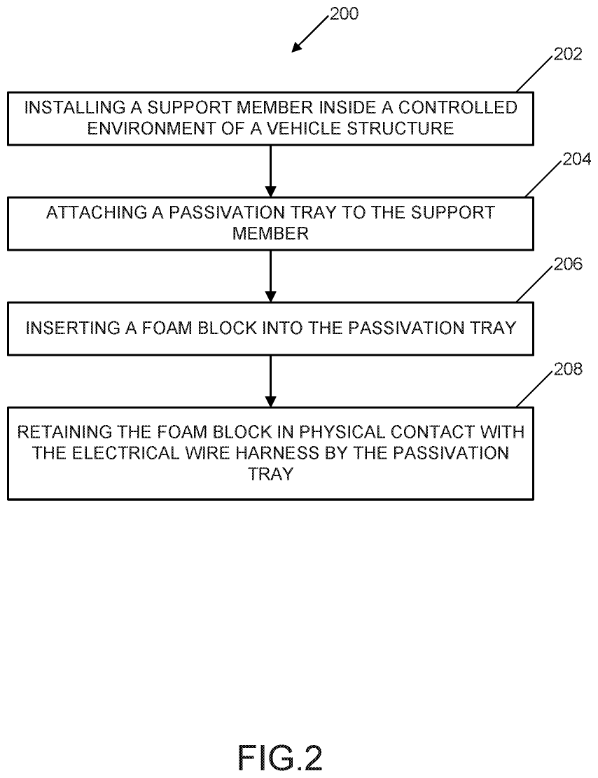

Reference is now made to FIG. 2 in addition to FIG. 1 to discuss a method 200 of passivating an electrical wire harness. In various embodiments, the method 200 may include installing a support member inside a controlled environment of a vehicle structure (block 202). The installing may be effectuated by attaching the support member to the vehicle structure with at least one attachment member. The method may include attaching a passivation tray to the support member (block 204). In various embodiments, the passivation tray includes at least one sheet metal flange. The method may include inserting a foam block into the passivation tray (block 206). The method may include retaining the foam block in physical contact with the electrical wire harness by the passivation tray (block 208). The foam block may be compressed by the passivation tray in compressive loading between the passivation tray and the vehicle structure.

In various instances, the inserting the foam block into the passivation tray is performed before the attaching the passivation tray to the support member. In other instances, the inserting the foam block into the passivation tray is performed after the attaching the passivation tray to the support member.

The inserting the foam block into the passivation tray at block 206 may also include interlocking a plurality of foam pieces having interlocking shapes to form the foam block, wherein the foam block comprises the plurality of foam pieces having interlocking shapes.

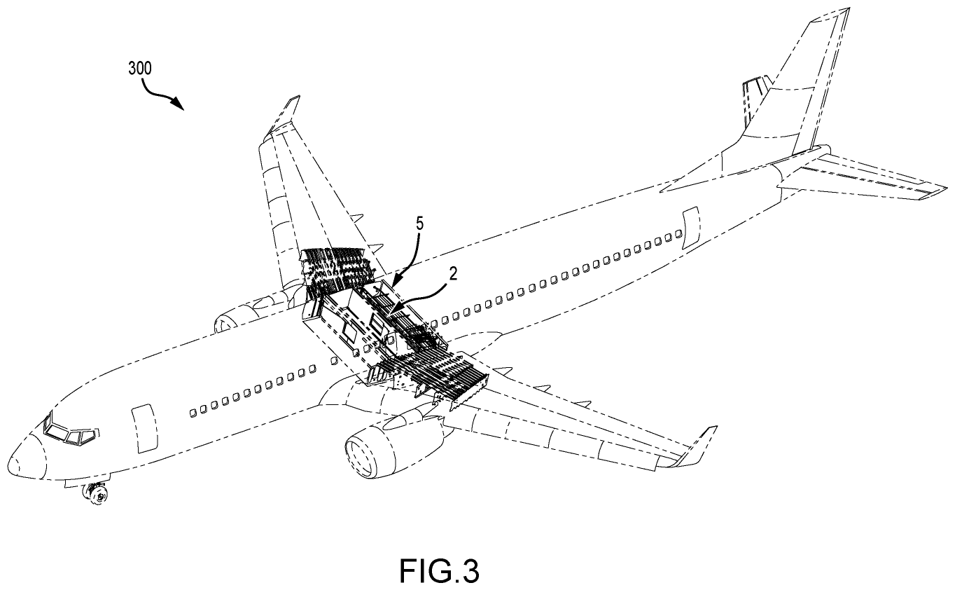

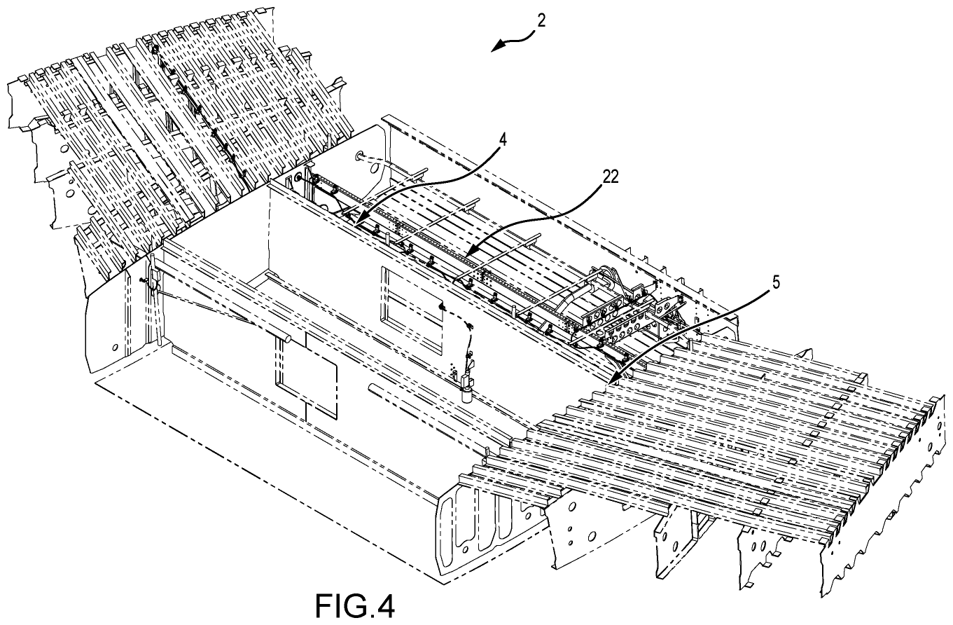

Referring to FIG. 3, an aircraft 300 is shown having a vehicle structure 5 that is a center fuel tank. A wiring passivation system 2 is installed in the vehicle structure 5. FIG. 4 shows a detailed view of aspects of the wiring passivation system 2 installed in the vehicle structure 5. A support member 22 is illustrated extending between two aspects of the vehicle structure 5 and attached thereto by attachment member 10 (not shown).

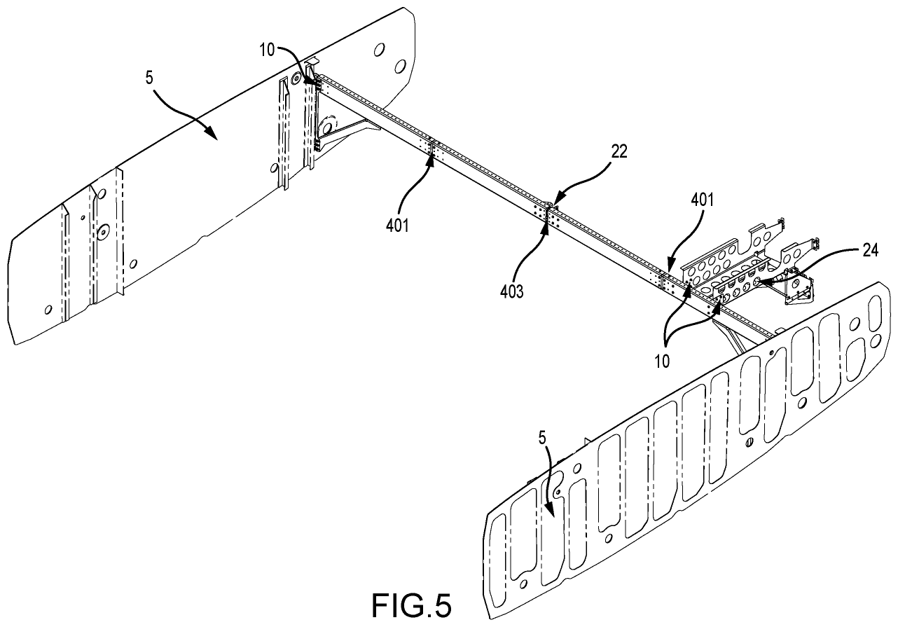

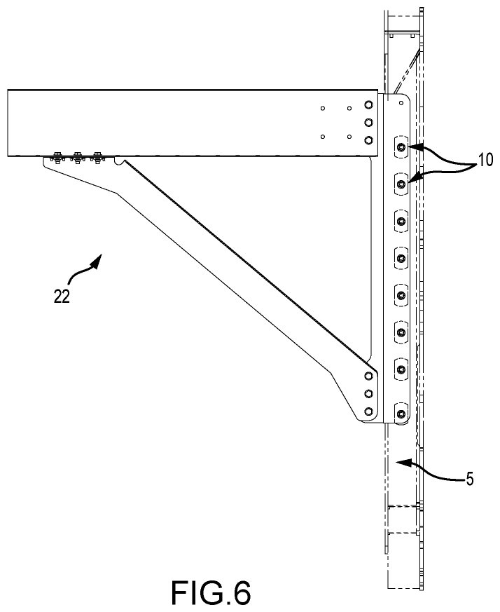

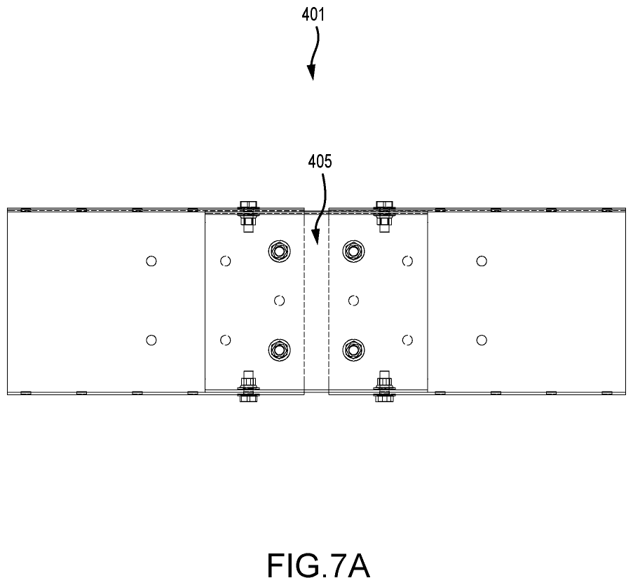

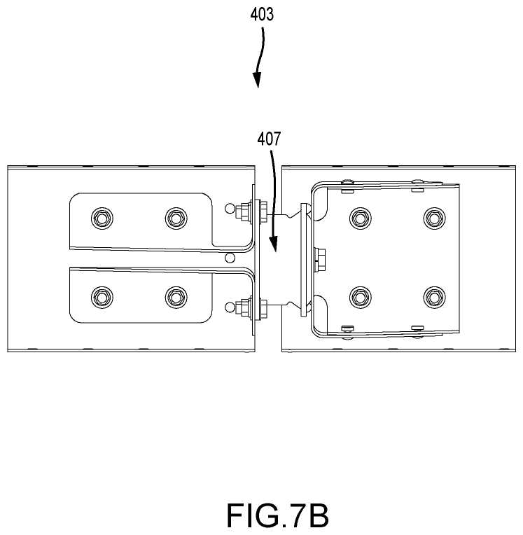

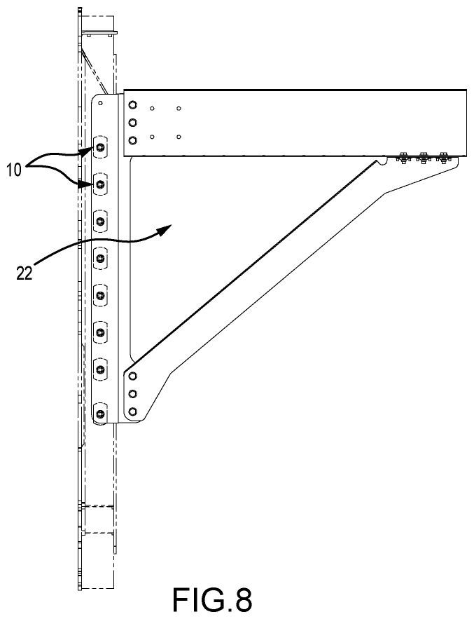

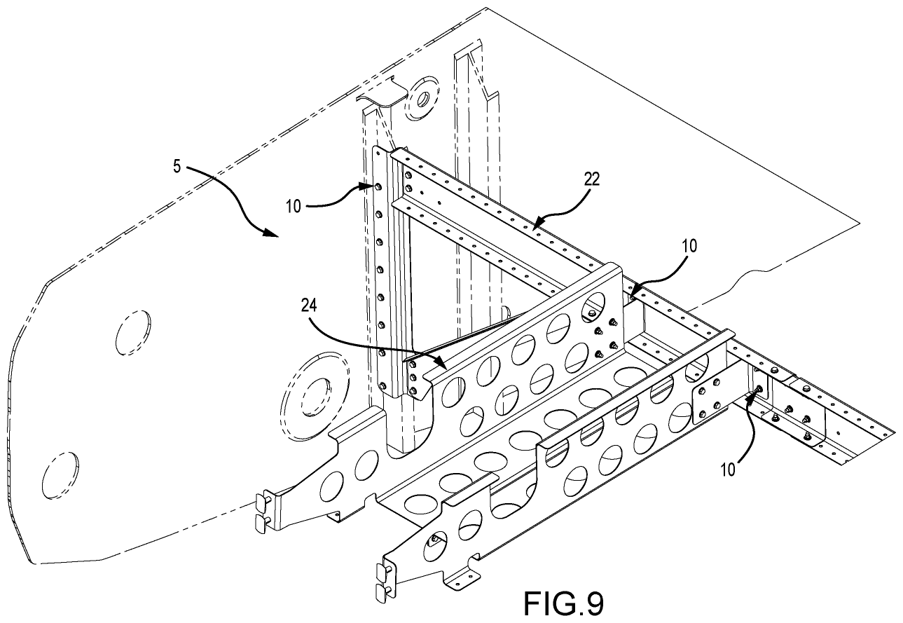

FIG. 5 shows a subset of features illustrated in FIG. 4 for additional clarity. FIG. 5 shows a support member 22 extending between two aspects of the vehicle structure 5, as well as a passivation tray 24 attached to the support member 22 by attachment members 10. FIG. 6 shows an end view of the support member 22 of FIG. 5. The support member 22 is attached to the vehicle structure 5 by attachment members 10. FIGS. 7A and 7B show detailed views of aspects of the support member illustrated in FIG. 5. For instance, a union 401 of two pieces may include channel feature 405 may be inserted into the support member 22 to provide a support member 22 made of multiple joined pieces. Moreover, an isolator feature 403 may provide a dielectric joint 407. The dielectric joint 407 may unite multiple joined pieces into a support member 22 of FIG. 5 without electrical conductivity between portions joining various aspects of the vehicle structure 5. FIG. 8 shows a similar view to that of FIG. 6 but from an opposite end of the support member 22. FIG. 8 shows an opposite end view of the support member 22 of FIG. 5. FIG. 9 shows a passivation tray 24 attached to the support member 22 by attachment members 10, as well as the support member 22 attached to the vehicle structure 5 by attachment members 10.

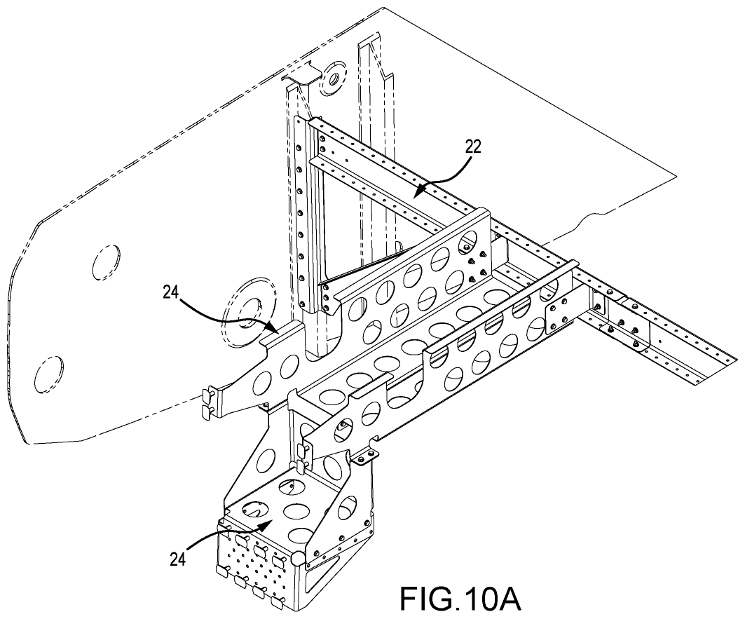

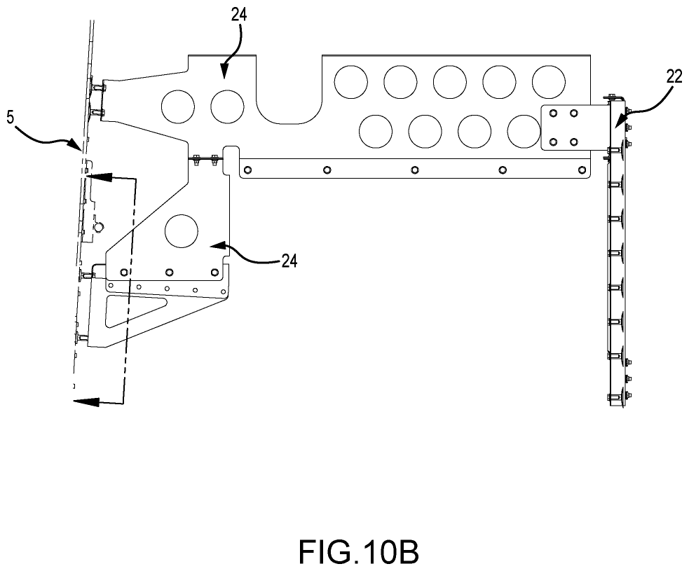

In various instances, multiple passivation trays 24 may be joined together to accommodate complex geometries. For instance, FIGS. 10A-B illustrates multiple passivation trays 24 attached together and supported by a same support member 22. In addition, a passivation tray 24 may abut or be attached with attachment members 10 directly to a vehicle structure 5, such as illustrated in FIG. 10B.

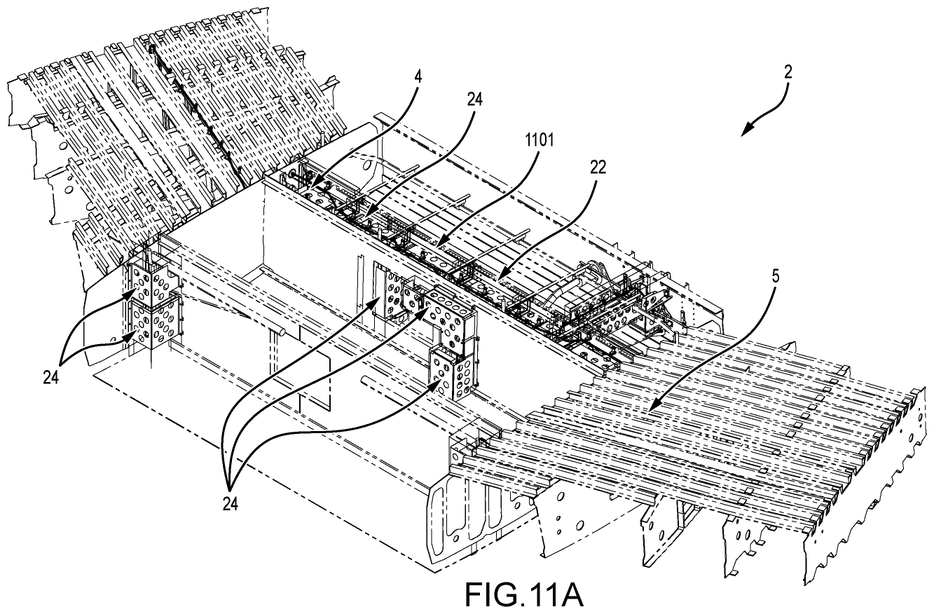

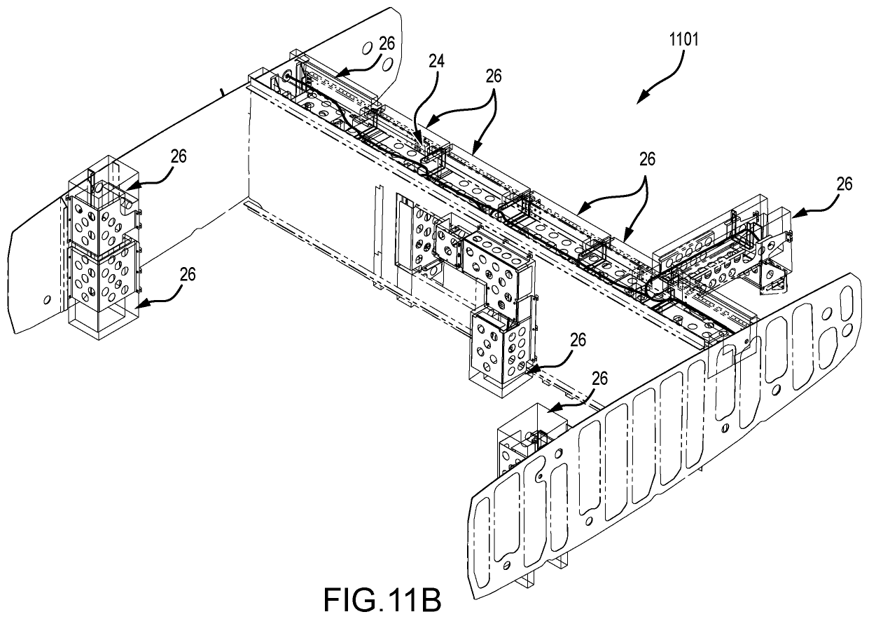

FIG. 11A shows a structure similar to that of FIG. 4, except with many passivation trays 24 installed. The support member 22 extends between aspects of a vehicle structure 5 and wiring 4 is illustrated in passivation trays 24 supported by the support member 22. Notably, various passivation trays 24 are connected directly to a vehicle structure 5. Thus, one may appreciate that the support member 22 may be omitted. Stated differently, the vehicle structure 5 may also be the support member 22 for passivation trays 24. FIG. 11A illustrates a first subassembly 1101 of passivation trays 24 that will be detailed further in subsequent figures.

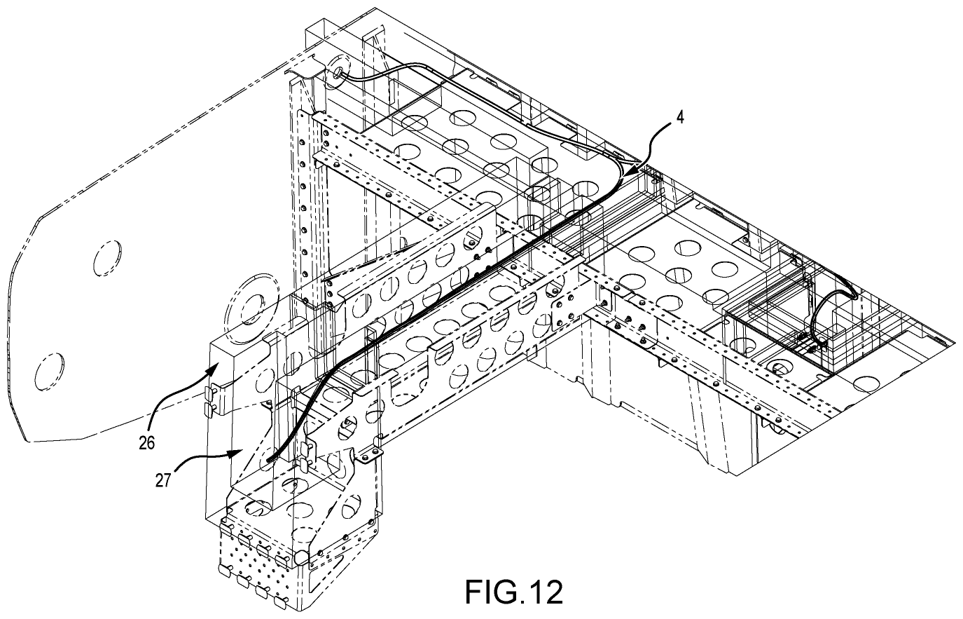

FIG. 11B shows the first subassembly 1101, with foam blocks 26 illustrated as transparent rectangles. FIG. 12 illustrates an example foam block 26 defining a channel 27 to receive wiring 4. Foam block 26 is illustrated having a channel 27 with a depth selected to provide isolation for the wiring 4 as the foam block 26 presses against a vehicle structure toward the upper end of the drawing, but yet accommodate space for wire movement, the foam block 26 being not being compressed against the wire. In further instances, a foam block may press against the wiring, pressing the wiring against a vehicle structure.

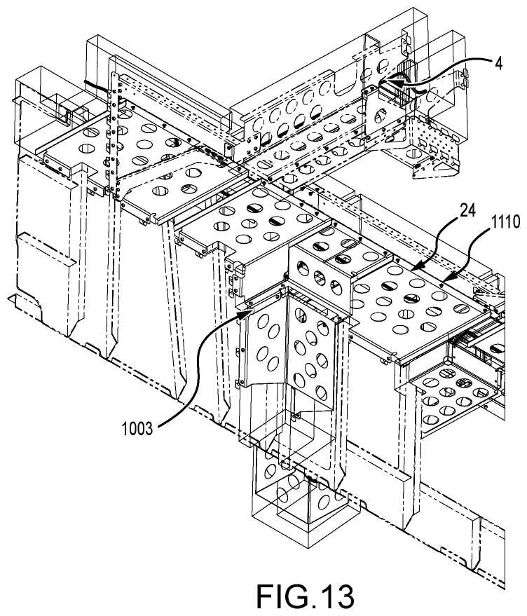

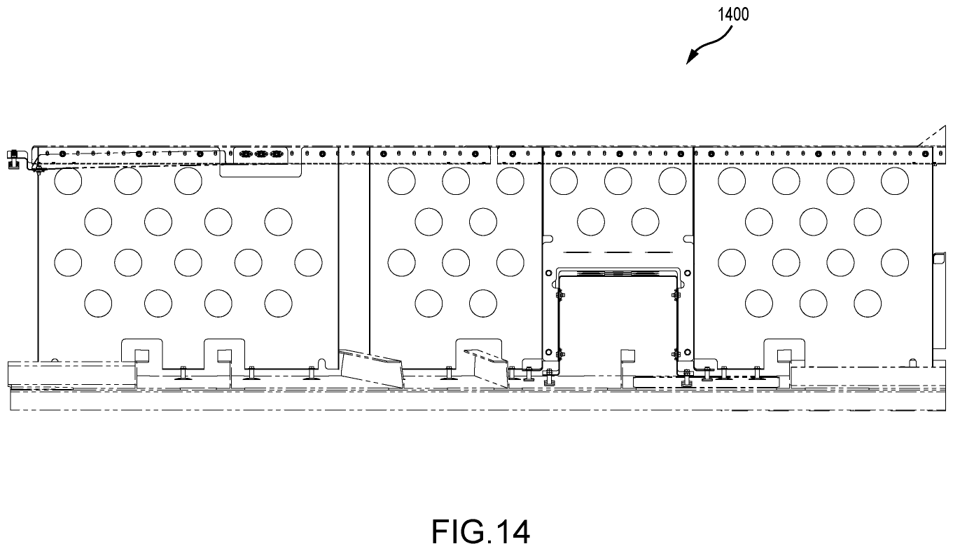

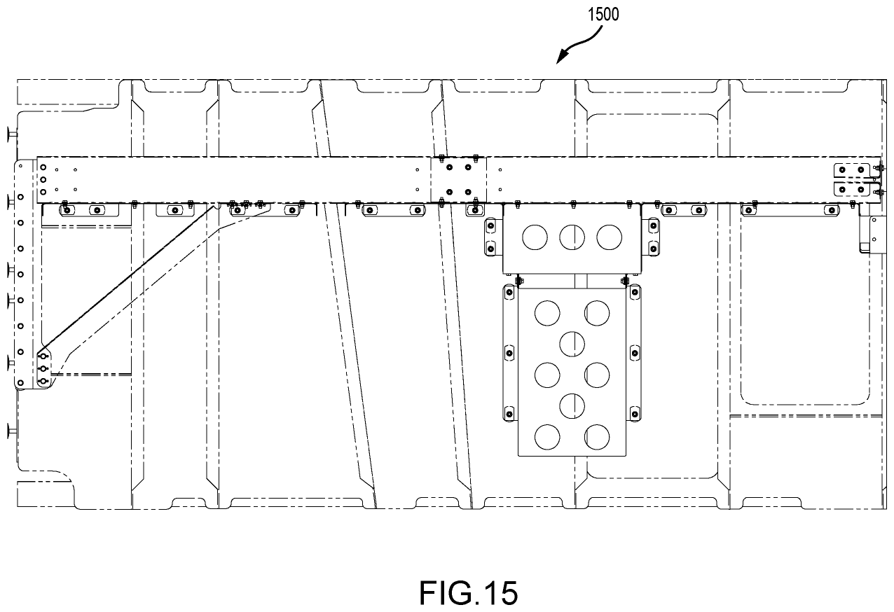

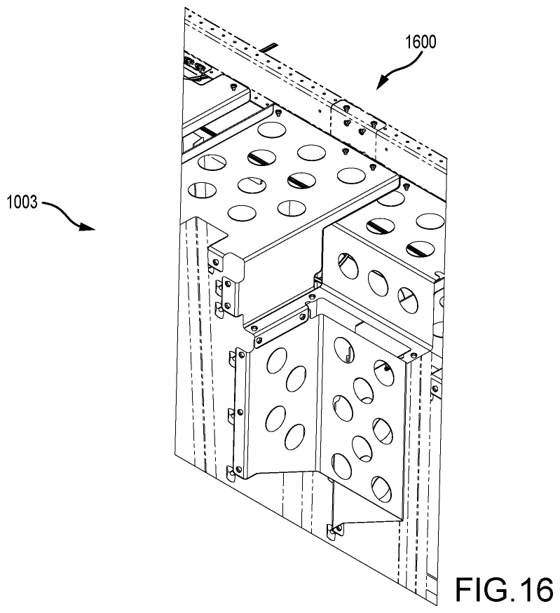

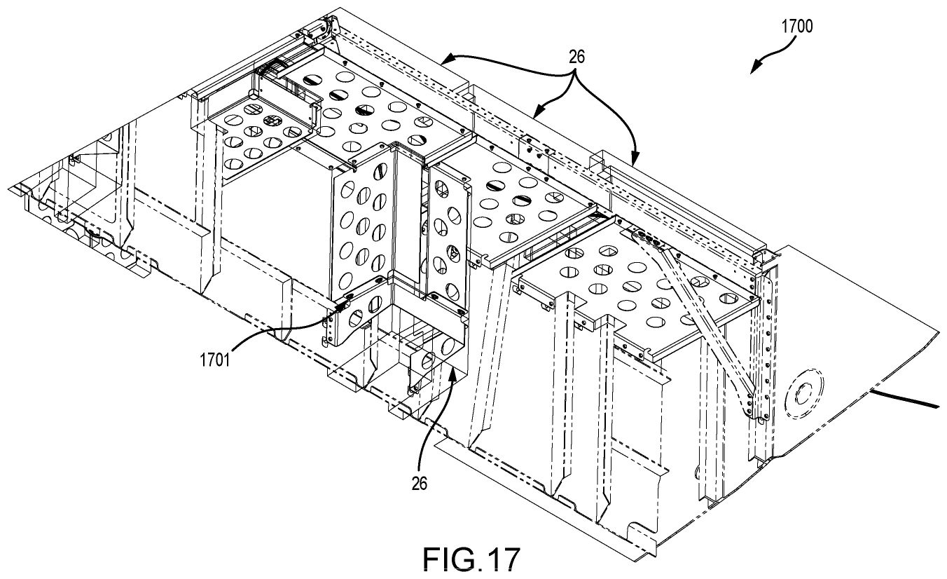

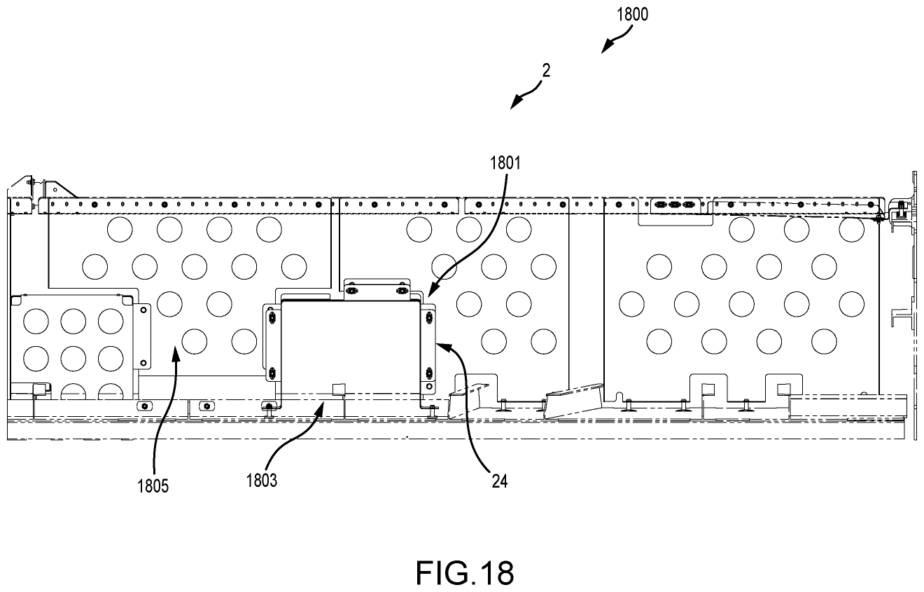

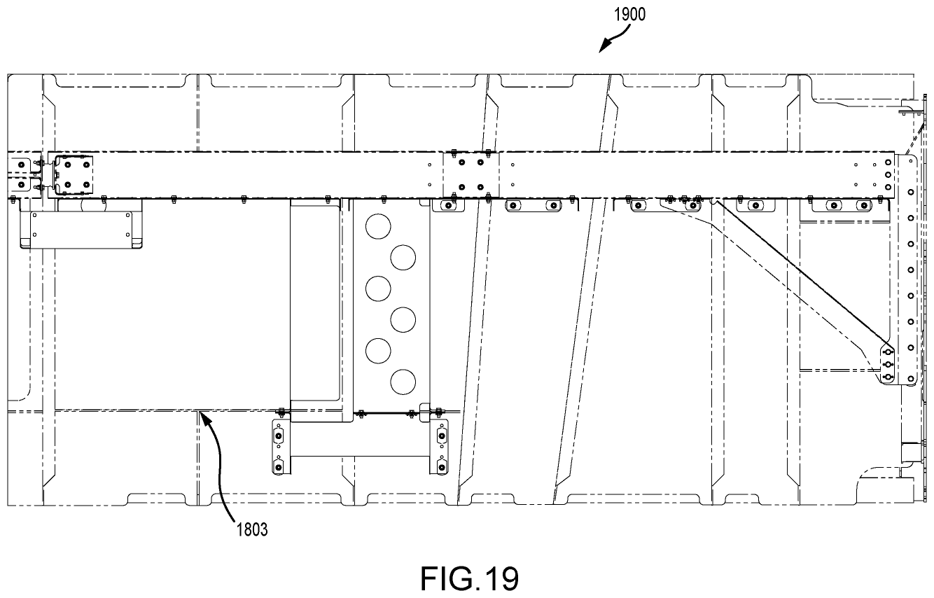

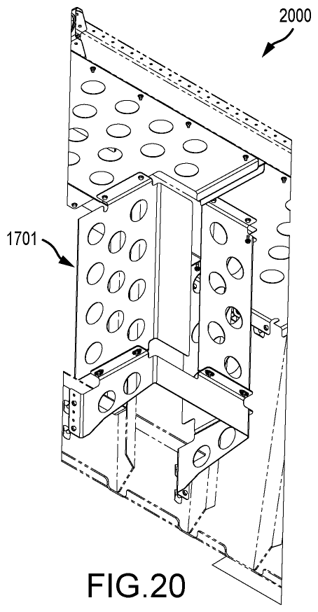

FIG. 13 illustrates the myriad arbitrary shapes available for passivation trays 24. FIG. 13 also illustrates details of the left-hand side foam installation. One may appreciate that complex geometries are accommodated across various aircraft components. One example configuration is illustrated in FIG. 13 having wiring extending in three different orthogonal directions. FIGS. 14-24 provide detailed views of other sections of passivation trays already disclosed herein. FIG. 14 shows an illustration 1400 of passivation trays 24 from FIG. 13 in a looking-up orientation. FIG. 15 shows an illustration 1500 of passivation trays 24 from FIG. 13 in a looking-forward orientation. FIG. 16 shows an illustration 1600 of passivation trays 24 from FIG. 13 detailing a tray-to-tray joint 1003 (FIG. 13). FIG. 17 shows an illustration 1700 having details of a portion of FIG. 11B, and in particular, installation of foam blocks 26. FIG. 18 shows a portion of the wiring passivation system 2 having a crawlspace 1803 through a bulkhead 1805. Various passivation trays 24 are illustrated in a stacked arrangement, to accommodate easy removal and replacement during maintenance operations necessitating access through the crawlspace 1803. FIG. 18 illustrates the view in a looking-up direction. FIG. 19 depicts an illustration 1900 of similar features as FIG. 18 but from a looking-forward direction and showing the crawlspace 1803 opening. FIG. 20 shows an illustration 2000 of assembly 1701 depicted in FIG. 17.

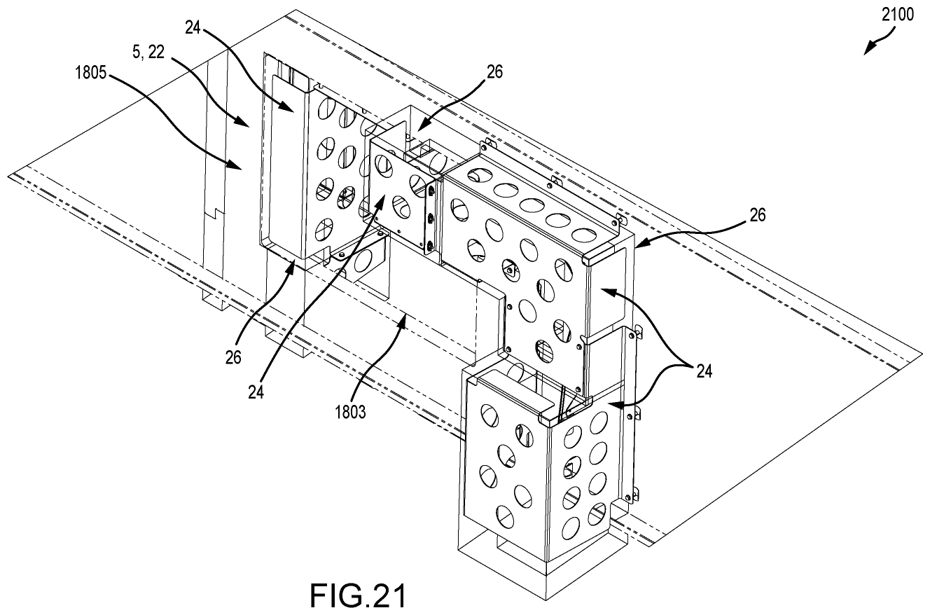

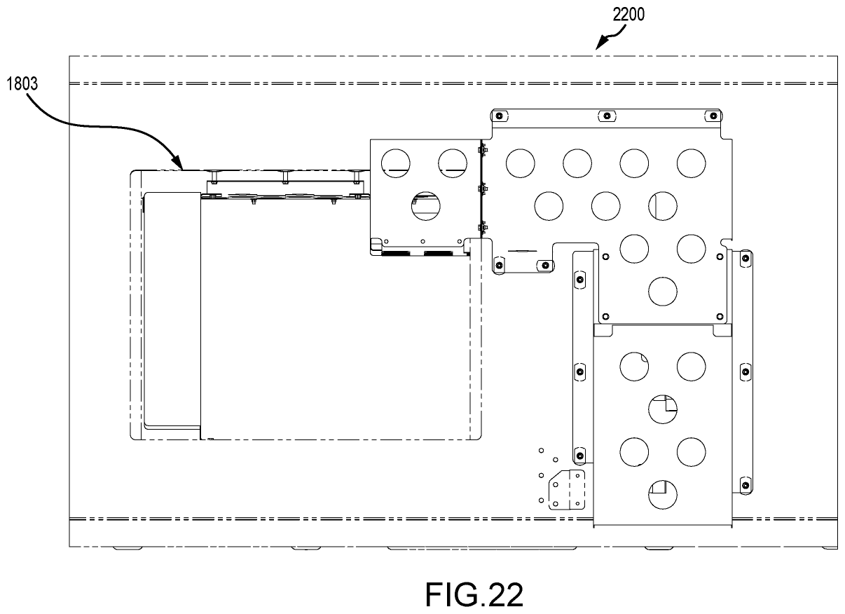

Directing attention now to FIG. 21, an illustration 2100 of the crawlspace 1803 through the bulkhead 1805 is shown in greater detail. Passivation trays 24 attach directly to the vehicle structure 5 serving as a support member 22. Foam blocks 26 are secured by the passivation trays 24 against the vehicle structure 5, e.g., a fuel tank internal bulkhead. Notably, while arranged to accommodate a wiring (not shown) passing through the bulkhead via the crawlspace 1830, the passivation trays 24 are attached with an arrangement facilitating ready removal for maintenance access through the crawlspace opening of the crawlspace 1803. FIG. 22 shows an illustration 2200 of aspects of the crawlspace 1803 but with the foam blocks omitted.

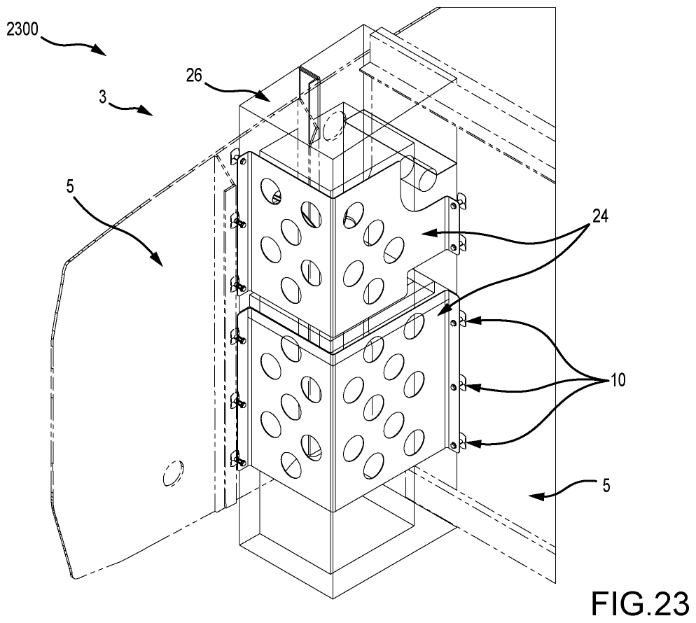

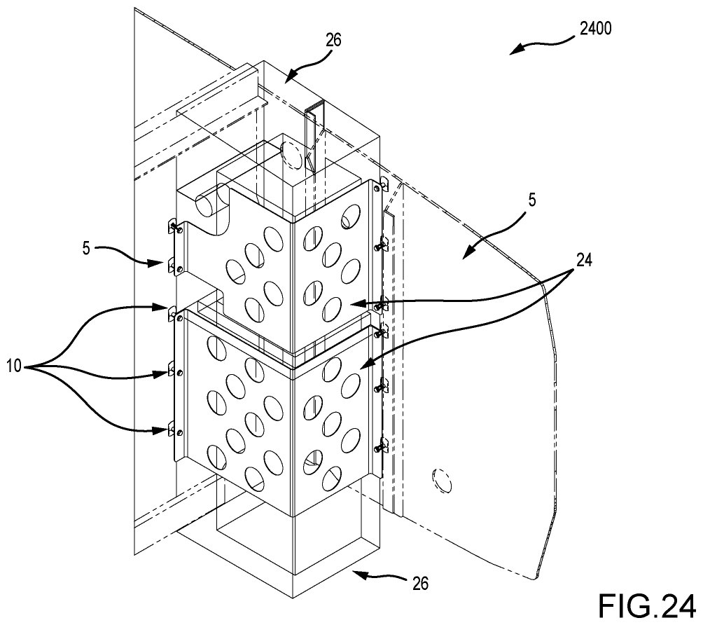

In various instances, fuel level sensing probes may be provided in an aircraft fuel tank. FIGS. 23 and 24 show detail views of various passivation trays 24 having a right-angle bent flange to accommodate passage of a wiring in a right-angle corner of a controlled environment 3 of a vehicle structure 5, such as to accommodate placement of a fuel level sensing probe in a controlled environment 3 comprising an aircraft fuel tank. As shown in FIG. 23 a vehicle structure 5 comprising two perpendicular bulkheads receives attachment members 10 and connects directly to a passivation tray 24 comprising a right-angle bent flange, to secure foam blocks 26 in the right-angle corner of the controlled environment 3 in connection with location of a fuel tank sensing probe.

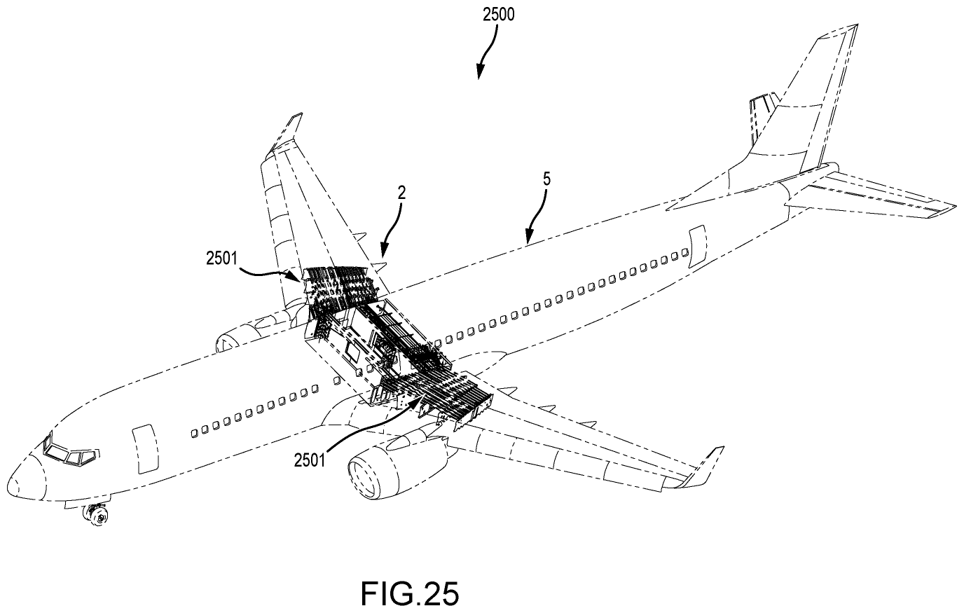

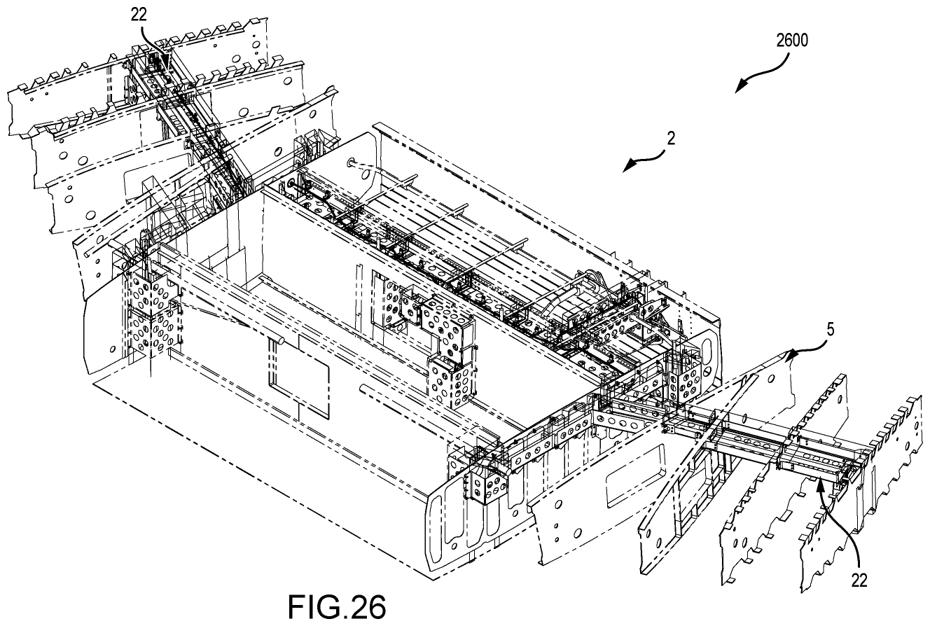

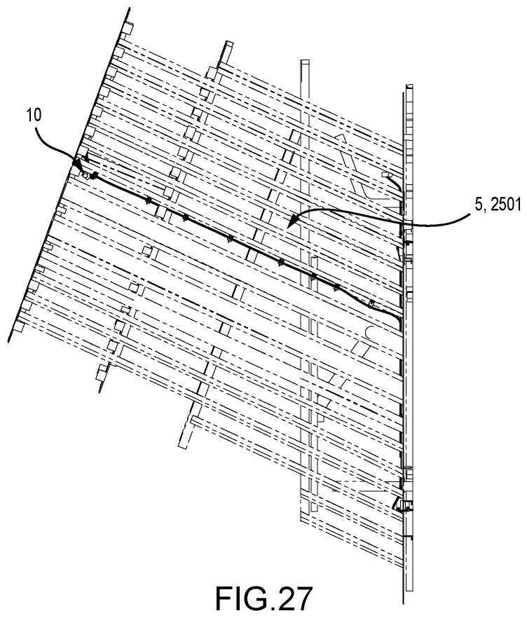

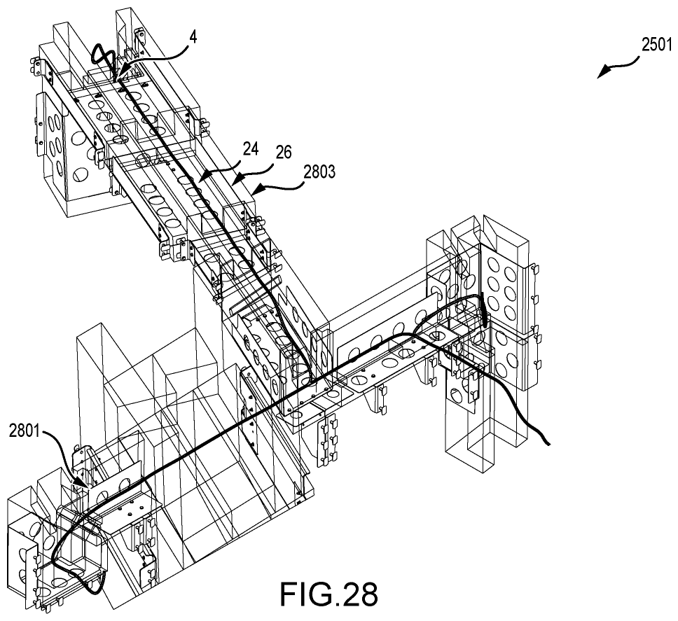

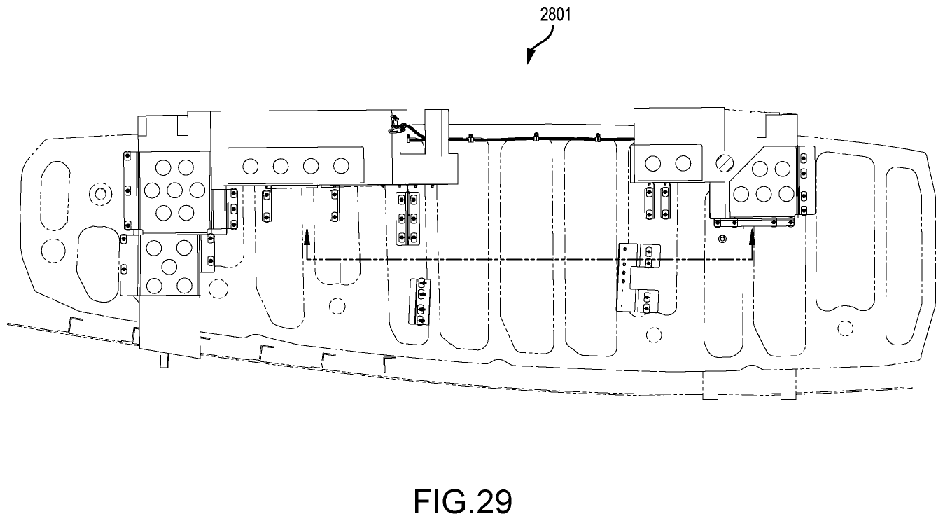

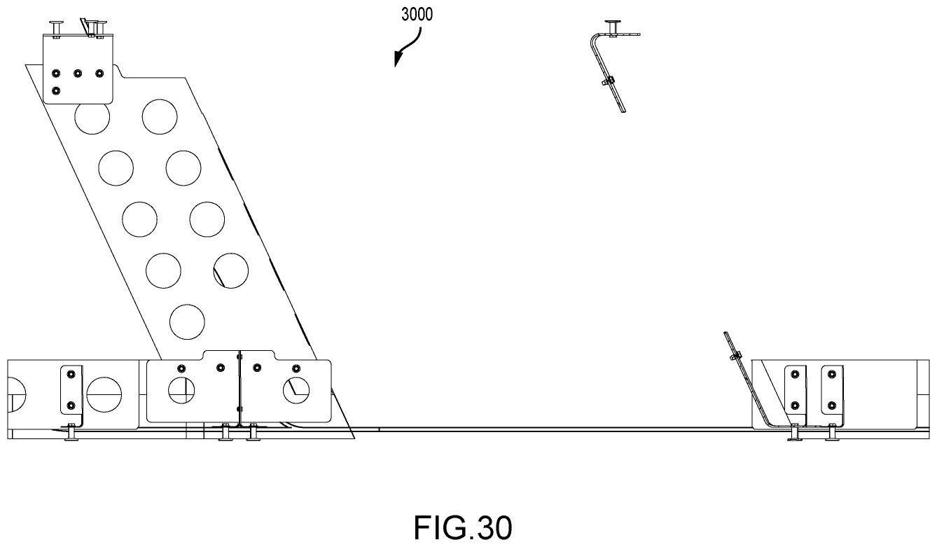

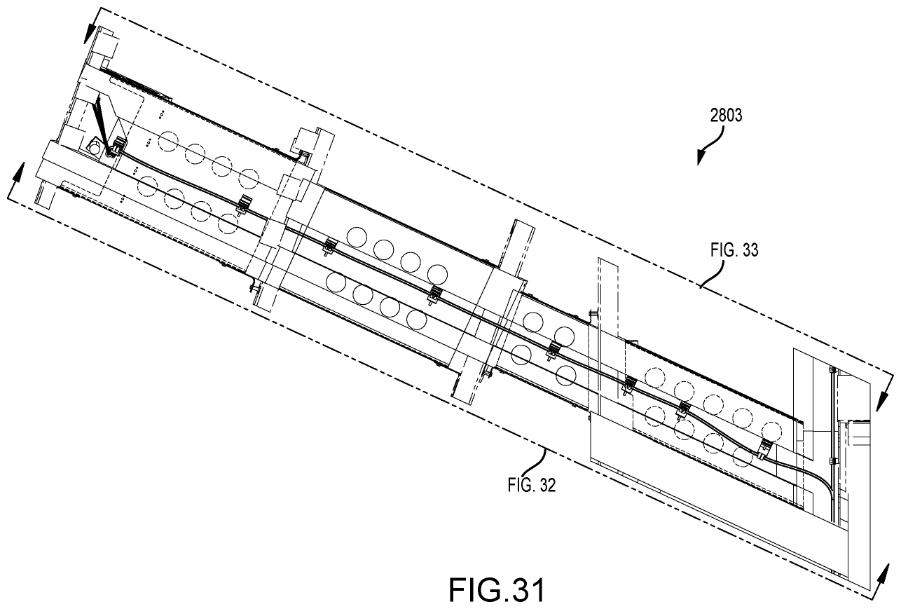

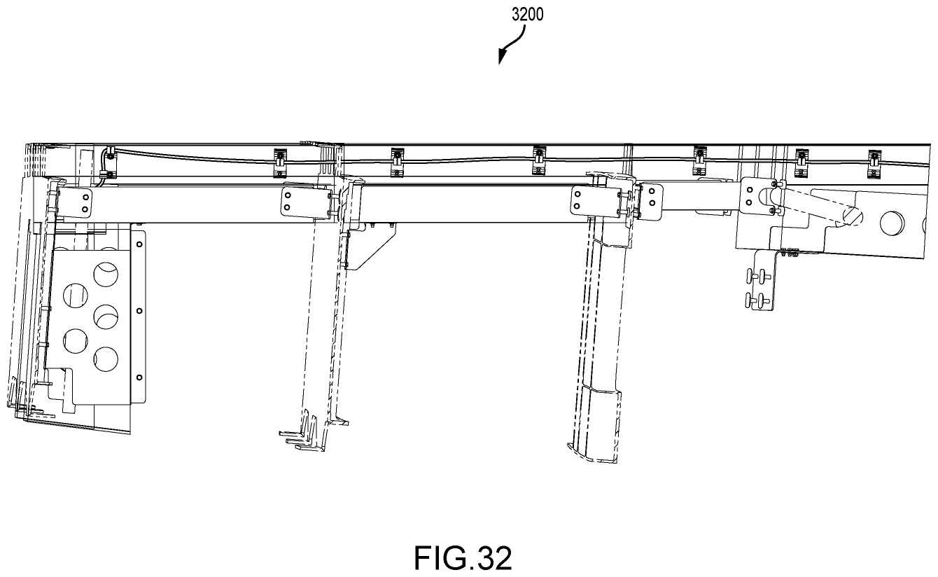

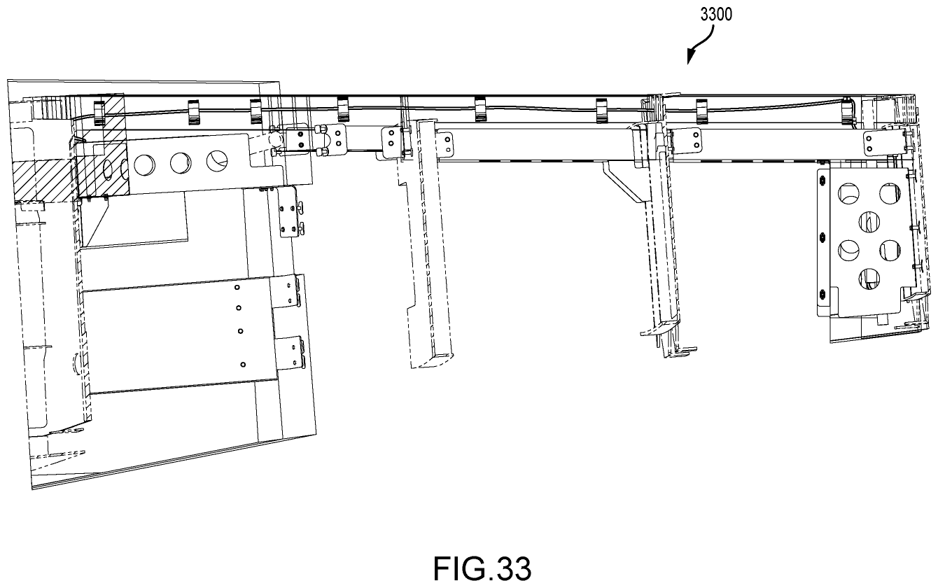

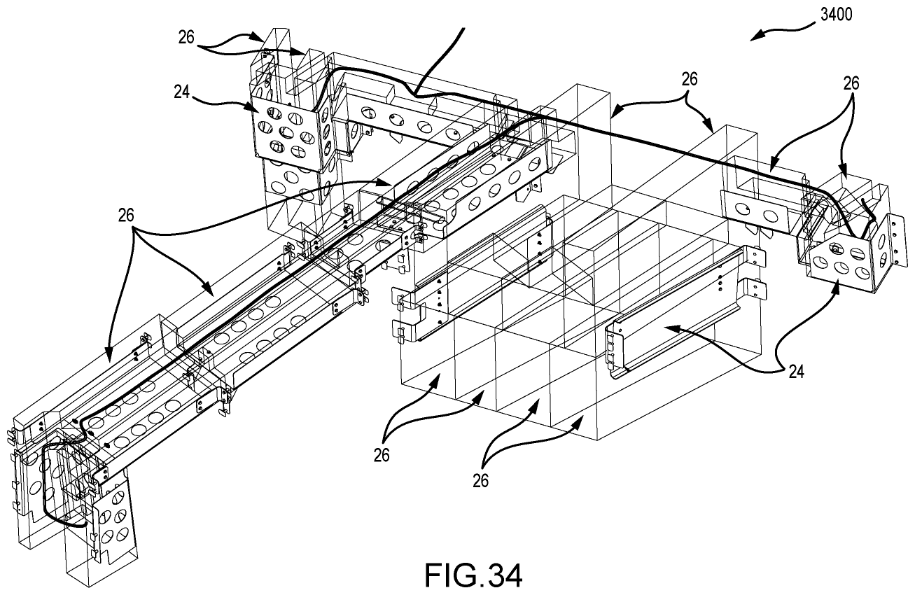

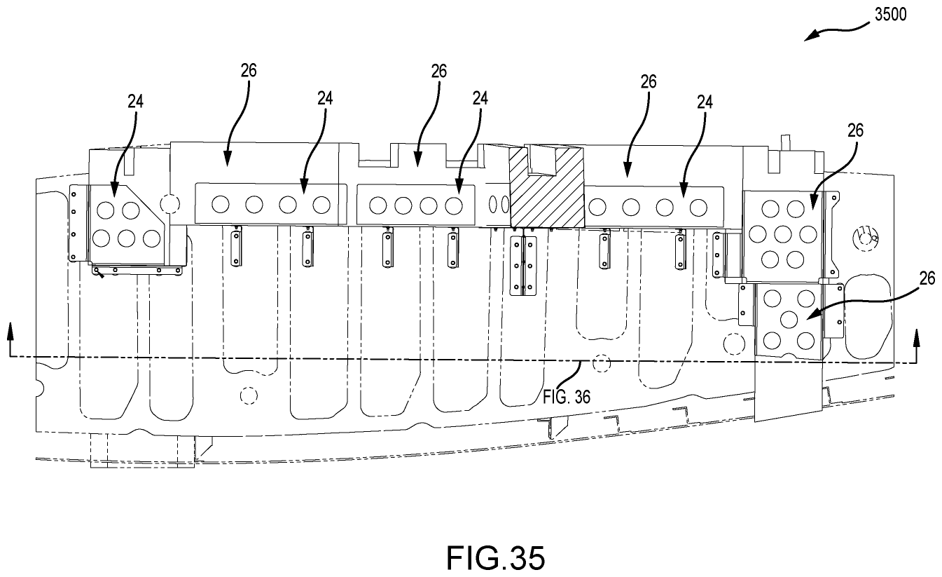

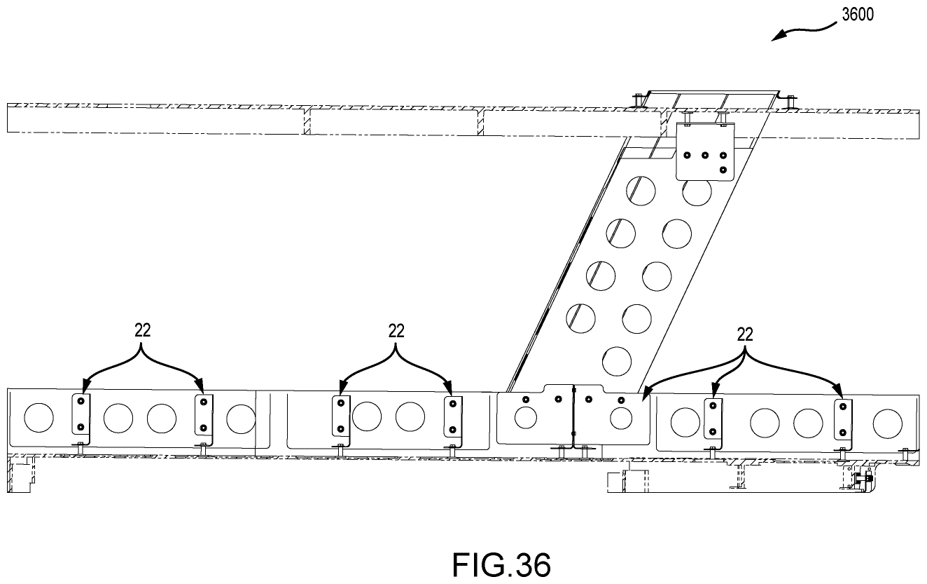

FIG. 25 shows an aircraft 2500 having a vehicle structure 5 that is a wing fuel tank 2501. Two wing fuel tanks 2501 are shown, one in each wing. A wiring passivation system 2 is installed in the vehicle structure 5. FIG. 26 shows a detailed view 2600 of aspects of the wiring passivation system 2 installed in the vehicle structure 5. A support member 22 is illustrated extending between two aspects of the vehicle structure 5 and attached thereto by attachment members 10 (not shown). FIG. 27 shows an electrical wire harness 4 with a probe extending through a vehicle structure 5 that is a wing fuel tank 2501. FIG. 28 provides a view of additional aspects of an electrical wire harness 4 extending through foam block 26 supported by passivation tray 24 in a wing fuel tank 2501. A forward/aft substructure 2801 is shown. An inboard/outboard substructure 2803 is shown FIG. 29 provides a detail view of the forward/aft substructure 2801. FIG. 30 shows a detail view 3000 of various other features including flanges and structures to attach passivation trays 24 in a desired location. FIG. 31 shows a detail view of the inboard/outboard substructure. FIG. 32 depicts a view 3200 of further features including flanges and structures to attach passivation trays 24 in desired locations shown from a looking-aft direction and FIG. 33 depicts a view 3300 of further features including flanges and structures to attach passivation trays 24 in desired locations shown from a looking-forward direction. FIG. 34 depicts a view 3400 of further features including flanges and structures to attach passivation trays 24 in connection with a float valve assembly for sensing a fuel level in a fuel tank. Notably, foam blocks 26 are depicted in a variety of sizes and with a variety of different contacting relations with associated passivation trays 24. desired locations shown from a looking-forward direction. FIG. 35 depicts a view 3500 showing yet further details of foam blocks 26 and passivation trays 24. Finally, FIG. 36 depicts a view 3600 showing gusset installations in connection with mounting the passivation trays. In various embodiments, gussets and/or clips may be a portion of the support members 22.

Benefits, other advantages, and solutions to problems have been described herein regarding specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods, and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is or is not explicitly recited in the claims. No claim element herein is to be construed under the provisions of 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Finally, any of the above-described concepts can be used alone or in combination with any or all the other above-described concepts. Although various embodiments have been disclosed and described, one of ordinary skill in this art would recognize that certain modifications would come within the scope of this disclosure. Accordingly, the description is not intended to be exhaustive or to limit the principles described or illustrated herein to any precise form. Many modifications and variations are possible considering the above teaching.

What is claimed is:

1. A wiring passivation system for passivating an electrical wire harness comprising:
a support member installable inside a controlled environment of a vehicle structure, the support member attachable to the vehicle structure with at least one attachment member;
a passivation tray comprising at least one sheet metal flange attachable to the support member; and
a foam block retained in physical contact with the electrical wire harness by the passivation tray, wherein the foam block is compressed by the passivation tray in compressive loading between the passivation tray and the vehicle structure.

2. The wiring passivation system according to claim 1, wherein the passivation tray is attached to the support member by a further at least one attachment member.

3. The wiring passivation system according to claim 2, wherein the further at least one attachment member is an electrically non-conductive mechanical connector.

4. The wiring passivation system according to claim 1, wherein the at least one attachment member is an electrically non-conductive mechanical connector.

5. The wiring passivation system according to claim 1, wherein the foam block comprises a plurality of foam pieces having interlocking shapes.

6. The wiring passivation system according to claim 1, wherein the passivation tray is a single piece of sheet metal having a plurality of bends.

7. The wiring passivation system according to claim 1, wherein the vehicle structure is an airplane.

8. The wiring passivation system according to claim 1, wherein the controlled environment is an airplane fuel tank.

9. A method of passivating an electrical wire harness, the method comprising:
installing a support member inside a controlled environment of a vehicle structure by attaching the support member to the vehicle structure with at least one attachment member;
attaching a passivation tray to the support member, the passivation tray comprising at least one sheet metal flange;
inserting a foam block into the passivation tray; and
retaining the foam block in physical contact with the electrical wire harness by the passivation tray, wherein the foam block is compressed by the passivation tray in compressive loading between the passivation tray and the vehicle structure.

10. The method of claim 9, wherein the inserting the foam block into the passivation tray is performed before the attaching the passivation tray to the support member.

11. The method of claim 9, wherein the inserting the foam block into the passivation tray is performed after the attaching the passivation tray to the support member.

12. The method of claim 9, further comprising interlocking a plurality of foam blocks having interlocking shapes to form the foam block, wherein the foam block comprises the plurality of foam blocks having interlocking shapes.

13. The method of claim 9, wherein the passivation tray is a single piece of sheet metal having a plurality of bends.

14. The method of claim 9, wherein the vehicle structure is an airplane.

15. The method of claim 9, wherein the controlled environment is an airplane fuel tank.

16. A fuel quantity indicating probe system for a vehicle fuel tank having a vehicle structure, the system comprising:
a support member attached by at least one attachment member to the vehicle structure and extending through a controlled environment comprising an interior space bounded by the vehicle structure, the interior space configured to retain a flammable fluid;
a passivation tray comprising at least one sheet metal flange sheet attached to the support member and providing a channel to receive a foam block;
a sensor to detect an amount of the flammable fluid in the interior space;
an electrical wire harness connected to the sensor to provide data or signals corresponding to the amount from the sensor to an indicator disposed outside of the controlled environment; and
the foam block, retained by the passivation tray in physical contact against the electrical wire harness, wherein the passivation tray presses the foam block in compressive loading between the passivation tray and the vehicle structure.

17. The fuel quantity indicating probe system according to claim 16, wherein the passivation tray is attached to the support member by a further at least one attachment member.

18. The fuel quantity indicating probe system according to claim 17, wherein the further at least one attachment member is an electrically non-conductive mechanical connector.

19. The fuel quantity indicating probe system according to claim 16, wherein the at least one attachment member is an electrically non-conductive mechanical connector.

20. The fuel quantity indicating probe system according to claim 16, wherein the foam block comprises a plurality of foam pieces having interlocking shapes.
